(19) United States
(12) Patent Application Publication
(10) Pub. No.: US 2021/0114967 A1
(43) Pub. Date: Apr. 22, 2021

(54) RESIST COMPOSITION FOR PATTERN PRINTING AND PATTERN FORMING METHOD

(71) Applicant: KANEKA CORPORATION, Osaka (JP)

(72) Inventors: Hitoshi TAMAI, Settsu-shi (JP); Masanori KANEMATSU, Settsu-shi (JP); Shimpei OKAMOTO, Settsu-shi (JP)

(73) Assignee: KANEKA CORPORATION, Osaka (JP)

(21) Appl. No.: 17/135,734

(22) Filed: Dec. 28, 2020

Related U.S. Application Data
(63) Continuation of application No. PCT/JP2019/025188, filed on Jun. 25, 2019.

(30) Foreign Application Priority Data
Jun. 29, 2018 (JP) 2018-124046

(51) Int. Cl. C07C 69/52; H01L 31/05; G03F 7/004; G03F 7/20; G03F 7/032; G03F 7/34

(52) U.S. Cl. CPC C07C 69/52; H01L 31/0516; G03F 7/34; G03F 7/2004; G03F 7/032; G03F 7/0045

(57) ABSTRACT

A resist composition for pattern printing contains a binder, a filler, a thickener, and a polyfunctional (meth)acrylate. The resist composition does not contain a photoinitiator. The resist composition also contains photocatalytic titanium oxide. A method for forming a pattern includes a resist composition that is pattern-wise printed, and then the resist composition is irradiated with an actinic radiation such that seepage of the resist component from an end of the pattern during the pattern formation using the resist composition is suppressed and the seepage portion is decomposed. As a result, it is possible to drastically reduce the seepage without impairing the rheology of the resist composition and additionally, to remove a slightly seeping portion without requiring, for example, a harmful ozone treatment or the like.

### CROSS-REFERENCE TO RELATED APPLICATIONS

This application claims benefit of priority to International Patent Application No. PCT/JP2019/025188, filed on Jun. 25, 2019, and to Japanese Patent Application No. 2018-124046, filed Jun. 29, 2018, the entire contents of each are incorporated herein by reference.

### BACKGROUND

### Technical Field

The present application relates to a resist composition for pattern printing, and a pattern forming method, and more particularly, to a resist composition for pattern printing and a pattern forming method for pattern printing which improve etching unevenness caused by seepage of a component at a pattern end during pattern formation using a resist composition.

### Background Art

Resist materials are used in a manufacturing process of semiconductors, plate making, printed circuit boards, or the like. Such resist materials are used in a treatment such as sand blasting, ion implantation, or etching such that a part of the surface of an object to be treated is protected by a resin or the like, and after the treatment, the protective film is peeled off, to thereby treat only a desired portion of the object. Thus, the resist materials are essential for manufacturing electronic components for home appliance applications, industrial applications, onboard applications, aeronautical applications and aerospace applications, industrial robots, solar batteries, or the like.

Etching resists has recently been required to achieve a high resolution with the thinning of wires in the circuit and the use of thinner circuits, and a resist material capable of wiring formation of 50 μm or less has been increasingly demanded. For the thinning of wires in the circuit, various properties are required, including not only fine wire printability of the resist material but also resistance to an etchant for a conductive layer and ease in peeling of the resist layer after etching.

In general, in the case of formation of an electrode of an electronic substrate or a solar battery electrode, an acidic etchant is mostly used for etching a conductive layer, and a resist to be applied is a resist of alkali development and peeling type. On the other hand, unlike photosensitive resists which requires exposure and development steps, in the case of pattern printing resists, there has been a problem that a binder component dissolved into a solvent from an end of a pattern seeps into the surface of a portion to be etched during a period of time from pattern formation to drying, and is firmly adhered in a subsequent drying step, and remains covering the surface of interest at the time of etching, resulting in uneven etching. A method has been known in which a layer for preventing hydrophobic solvent permeation is first formed on a base material using a material such as polysiloxane and fluorinated polysiloxane having a two-dimensional crosslinked structure or a metal layer that has been surface-treated with the material for hydrophobization, and a resist pattern is formed on top of the layer to thereby prevent the seepage, and then the layer for preventing hydrophobic solvent permeation is removed with ozone, which has an ability to decompose organic substances (see JP2016-10965A). However, because a treatment in water is necessary and such a treatment is complicated, and the removal ability of the treatment is unsatisfactory, the method has actually not been necessarily applied to a production process. Further, as a measure for improving the pattern printing resist itself, a resist composition has also been proposed in which the amount of a solvent component which is one of the causes of the seepage is reduced and a phenol resin is used as a main resin component, and a thickener is further incorporated (see JP2004-260143A). However, such a resist composition raises a concern that the rheology of the resist is impaired, and because complete elimination of the solvent is impossible, the seepage has not been completely prevented.

### SUMMARY

The present application provides a resist composition for pattern printing and a pattern forming method which improve etching unevenness caused by the seepage of a component at an end of a pattern during pattern formation using a resist composition. In addition, it is a further object of the present application to provide a resist composition for pattern printing and a pattern forming method which drastically reduce the seepage of the resist without impairing the rheology of a resist, unlike the case of the measure through the solvent reduction in a resist composition, and also enables a slightly seeping portion to be removed without requiring, for example, a harmful ozone treatment or the like.

In order to solve the above problems, the present inventors have conducted extensive studies, and have found that as a basic design of a resist composition, a polyfunctional (meth)acrylate is blended into a resist composition without blending a photoinitiator, from the viewpoint that the reduction in amount of the solvent is not merely relied on as a method for suppressing the seepage and a thin film sufficient to suppress the flow of a resist pattern-developed by printing is formed on the resist surface after pattern printing using a resist composition through irradiation with an actinic radiation such as ultraviolet light (UV). Further, the present inventors have found that removal of the seepage generated in a short time period from printing to UV exposure without using a harmful component such as ozone based on the photodecomposition effect of the photocatalyst blended in the resist composition enables more excellent etching performance to be maintained, thereby to complete the present application.

A first aspect of the present application for solving the above problems relates to a resist composition for pattern printing, containing a binder component, a filler component, a thickener component, and a polyfunctional (meth)acrylate, and not containing a photoinitiator.

In addition, in one embodiment of the first aspect, the resist composition for pattern printing is described, in which the number of (meth)acrylate functional groups in the polyfunctional (meth)acrylate is at least 3 or more.

In a further embodiment of the first aspect, the resist composition for pattern printing is described, in which the polyfunctional (meth)acrylate is blended in an amount of 0.1 parts by mass or more and 10 parts by mass or less with respect to 100 parts by mass of the resist composition for pattern printing.

In one embodiment of the first aspect, the resist composition for pattern printing is also described, in which the resist composition for pattern printing further contains a photocatalyst component.

In one embodiment of the first aspect, the resist composition for pattern printing is also described, in which the photocatalytic component contains titanium oxide having photocatalytic activity.

In one embodiment of the first aspect, the resist composition for pattern printing is further described, in which the average particle diameter of the titanium oxide is 1 nm or more and 200 nm or less.

In one embodiment of the first aspect, the resist composition for pattern printing is described, in which the content of the titanium oxide is 0.01 parts by mass or more and 5 parts by mass or less with respect to the total 100 parts by mass of the resist composition for pattern printing.

A second aspect of the present application for solving the above problems relates to a method for producing a solar battery cell, including:

a printing step of printing a resist pattern on a substrate having a semiconductor layer using a resist composition for pattern printing containing a binder component, a filler component, a thickener component, and a polyfunctional (meth)acrylate, and not containing a photoinitiator; an etching step of etching a part of the semiconductor layer and a part of the resist pattern; and a peeling step of peeling off the residual resist pattern.

In one embodiment of the second aspect, the method for producing a solar battery cell is described, in which the resist pattern is irradiated with an actinic radiation after the printing step prior to the etching step, or in the etching step.

In one embodiment of the second aspect, the method for producing a solar battery cell is further described, in which the actinic radiation is an ultraviolet radiation or an electron beam.

In addition, in one embodiment of the second aspect, the method for producing a solar battery cell is described, in which the resist composition for pattern printing further contains a photocatalyst component.

Further, in one embodiment of the second aspect, the method for producing a solar battery cell is described, in which in the etching step, one or more selected from the group consisting of an aqueous acid solution, an aqueous alkaline solution, and water are brought into contact with the semiconductor layer and/or the resist pattern during or after the irradiation with the actinic radiation.

In one embodiment of the second aspect, the method for producing a solar battery cell is described, in which in the peeling step, an aqueous alkaline solution or an alcohol-containing aqueous solution is brought into contact with the semiconductor layer and/or the resist pattern.

In one embodiment of the second aspect, the method for producing a solar battery cell is also described, in which the actinic radiation is applied even during or after the peeling step to remove a resist peeling residue.

A third aspect of the present application for solving the above problems relates to a solar battery including a solar battery cell which is produced by the method for producing a solar battery cell according to the second aspect of the present application.

According to the present application, a polyfunctional (meth)acrylate is blended in a resist composition for pattern printing but a photoinitiator is not blended, and this makes it possible to extremely and effectively suppress seepage of a resist component at an end of the pattern during pattern formation without impairing the rheology of the resist and to suppress the etching unevenness caused by the seepage.

In addition, according to one preferred embodiment of the present application, further blending of a photocatalyst such as titanium oxide having photocatalytic activity into the resist composition for pattern printing enables a portion slightly seeping at the end of the pattern during pattern formation to be removed by immersion in water or the like without a need of a harmful ozone treatment or the like, which allows a further excellent etching performance to be exhibited.

### DETAILED DESCRIPTION

One embodiment of the present application will be described below, but the present application is not limited thereto.

### [Resist Composition for Pattern Printing]

The resist composition for pattern printing according to the present application contains a binder component, a filler component, a thickener component, and a polyfunctional (meth)acrylate, and does not contain a photoinitiator.

Because the resist composition for pattern printing according to the present application has the above-described composition, only the surface portion of the resist pattern printed using the resist composition for pattern printing according to the present application can be cured by means of irradiation with the actinic radiation after the pattern printing to form a film, and therefore the flow of the resist developed in a predetermined pattern shape can be suppressed and the seepage from the resist component at an end of the resist pattern can be reduced. The resist composition for pattern printing of the present application has been designed in consideration of the following points.

Specifically, in general, when a polyfunctional (meth)acrylate which is a crosslinking agent component is added to a resist composition, an initiator component such as a photoinitiator which triggers a reaction is usually used in combination with the crosslinking agent. However, with regard to the resist composition for pattern printing according to the present application, the film thickness of the pattern at the time of printing is assumed to be on the order of less than a millimeter; thus, when such an initiator component is present, crosslinking also proceeds inside the resist pattern upon irradiation with an actinic radiation, and the peeling performance is lowered. Therefore, a composition in which the photoinitiator is not blended is adopted, and it is envisioned that an oxidative crosslinking of a polyfunctional (meth)acrylate component by the irradiation with the actinic radiation such as ultraviolet light (UV) is utilized to form a thin film only on the surface of the resist layer developed in a pattern, thereby suppressing the flow of the resist from the predetermined pattern and preventing the seepage.

Further, in a preferred embodiment of the resist composition for pattern printing according to the present application, a photocatalytic component such as titanium oxide having photocatalytic activity is blended into the composition. This preferred embodiment of the resist composition for pattern printing of the present application has been designed in consideration of the following points.

Specifically, because the photocatalytic component such as titanium oxide has an ability to decompose an organic substance in the presence of light, the photocatalytic component is not generally used in a composition such as a coating agent and a resist agent, which contains an organic binder. However, with regard to the resist composition for pattern printing according to the present application, as a result of extensive studies, the photocatalyst component is added into the resist composition contrary to the conventional common general knowledge in the art based on the following viewpoint: when a region of the original resist pattern portion developed into a predetermined pattern is irradiated with an actinic radiation (UV, in particular) after pattern printing, the binder at a certain concentration is present in the region, and thus the presence of such a photocatalyst component in the resist composition does not affect main performances including etchant resistance, whereas in the thin layer region of the portion that has seeped from the end of the resist pattern, the ability of the photocatalyst component to decompose an organic substance is exhibited to only decompose the seepage portion suitably.

Hereinafter, each component will be described in sequence.

### [Binder Component]

The binder component of the present application is not particularly limited as long as it is used in a resist agent. Specific examples include organic binders such as an acrylic resin, a phenol formaldehyde resin, an epoxy resin, a urethane resin, a silicone resin, paravinylphenol, a polyvinylpyrrolidone resin, and a polyvinyl alcohol resin, and these can be used alone or in a combination of a plurality thereof. Among these resins, an acrylic resin, a phenol formaldehyde-based resin, a urethane-based resin, and a silicone-based resin are preferred, and an acrylic resin and a phenol formaldehyde-based resin are more preferred from the viewpoint of balance between the rheology, etchant resistance and cost of a resist.

### <Acrylic Resin>

The monomer constituting the acrylic resin is not particularly limited, and examples thereof include (meth)acryloyl-based monomers such as methyl (meth)acrylate, ethyl (meth)acrylate, propyl (meth)acrylate, butyl (meth) acrylate, pentyl (meth) acrylate, hexyl (meth) acrylate, heptyl (meth)acrylate, octyl (meth)acrylate, and 2-ethylhexyl (meth)acrylate. These monomers may be used alone or in a combination of two or more thereof.

In addition, in one embodiment of the resist composition for pattern printing according to the present application, it is preferable that the acrylic resin used as a binder resin includes a structural unit derived from a polymerizable compound having an ether bond. When the structural unit is included, favorable adhesion to the substrate during development, and favorable plating solution resistance may be exhibited.

The monomer having an ether bond is not particularly limited, and examples thereof include radically-polymerizable compounds such as (meth)acrylic acid derivatives having an ether bond and an ester bond including 2-methoxyethyl (meth) acrylate, 2-ethoxyethyl (meth) acrylate, methoxy triethylene glycol (meth)acrylate, 3-methoxybutyl (meth) acrylate, ethylcarbitol (meth) acrylate, phenoxy polyethylene glycol (meth)acrylate, methoxy polypropylene glycol (meth)acrylate, and tetrahydrofurfuryl (meth)acrylate. 2-Methoxyethyl (meth)acrylate, 2-ethoxyethyl (meth)acrylate, and methoxy triethylene glycol (meth)acrylate are preferred. These monomers may be used alone or in a combination of two or more thereof.

Additionally, the following monomer having an ethylenic unsaturated double bond may be copolymerized. Examples of such a monomer include: acrylamides such as diacetone acrylamide; acrylonitrile; ethers of vinyl alcohol such as vinyl n-butyl ether; (meth)acrylic acid-based monomers such as alkyl (meth) acrylates, tetrahydrofurfuryl (meth) acrylate, dimethylaminoethyl (meth) acrylate, diethylaminoethyl (meth)acrylate, glycidyl (meth)acrylate, 2,2,2-trifluoroethyl (meth)acrylate, 2,2,3,3-tetrafluoropropyl (meth)acrylate, (meth)acrylic acid, α-bromo(meth)acrylic acid, α-chloro(meth)acrylic acid, β-furyl(meth)acrylic acid, and β-styryl(meth)acrylic acid; maleic acid-based monomers such as maleic acid, maleic anhydride, monomethyl maleate, monoethyl maleate, and monoisopropyl maleate; fumaric acid; cinnamic acid; α-cyanocinnamic acid; itaconic acid; crotonic acid; propiolic acid; and the like. These may be used alone or in any combination of two or more thereof.

Although not essential for the resist, further copolymerizing a monomer having a carboxyl group may allow for resist peeling using an aqueous alkaline solution.

Examples of the monomer having a carboxyl group include: monocarboxylic acids such as acrylic acid, methacrylic acid, and crotonic acid; dicarboxylic acids such as maleic acid, fumaric acid, and itaconic acid; compounds having a carboxyl group and an ester bond such as 2-methacryloyloxyethylsuccinic acid, 2-methacryloyloxyethylmaleic acid, 2-methacryloyloxyethylphthalic acid, and 2-methacryloyloxyethylhexahydrophthalic acid; and the like, and acrylic acid and methacrylic acid are preferred. These monomers may be used alone or in a combination of two or more thereof.

In addition, in the present application, the mass average molecular weight in terms of polystyrene by gel permeation chromatography (hereinafter, referred to as “GPC”) (hereinafter, “mass average molecular weight in terms of polystyrene by GPC” is simply referred to as “mass average molecular weight”) of the acrylic resin is preferably in a range of 230,000 to 600,000, and particularly desirably in a range of 240,000 to 500,000. When the mass average molecular weight is 230,000 or more, excellent stress resistance to plating is exhibited, the metal layer obtained by plating treatment is less likely to bulge, leading to a favorable shape of the product obtained by plating, excellent plating solution resistance is also exhibited, and chipping or cracking less likely to occurs in the resist during the plating treatment or washing after the plating process, and therefore the mass average molecular weight of 230,000 or more is preferable. Further, when the mass average molecular weight is 600,000 or less, peelability of an unexposed portion of the resist from the substrate tends to be improved, and therefore the mass average molecular weight of 600,000 or less is preferable.

### <Phenol Formaldehyde Resin>

The phenol formaldehyde resin is obtained, for example, by polymerizing phenols and aldehydes in a ketone-based solvent in the presence of a catalyst. Although the resin is classified into a novolac resin which is polymerized under acidic conditions and a resol resin which is polymerized under alkaline conditions, a novolac resin having a linear structure is preferred from the viewpoint of ensuring resist performances more reliably. The phenol formaldehyde resin may be used alone or in a combination of two or more thereof.

Examples of the phenols include: phenol; cresols such as m-cresol, p-cresol, and o-cresol; xylenols such as 2,3-xylenol, 2,5-xylenol, 3,5-xylenol, and 3,4-xylenol; alkylphenols such as m-ethylphenol, p-ethylphenol, o-ethylphenol, 2,3,5-trimethylphenol, 2,3,5-triethylphenol, 4-tert-butylphenol, 3-tert-butylphenol, 2-tert-butylphenol, 2-tert-butyl-4-methylphenol, and 2-tert-butyl-5-methylphenol; alkoxyphenols such as p-methoxyphenol, m-methoxyphenol, p-ethoxyphenol, m-ethoxyphenol, p-propoxyphenol, and m-propoxyphenol; isopropenylphenols such as o-isopropenylphenol, p-isopropenylphenol, 2-methyl-4-isopropenylphenol, and 2-ethyl-4-isopropenylphenol; arylphenols such as phenylphenol; polyhydroxyphenols such as 4,4′-dihydroxybiphenyl, bisphenol A, resorcinol, hydroquinone, and pyrogallol; and the like. Among these, m-cresol and p-cresol are particularly preferred. The phenols may be used alone or in a combination of two or more thereof.

Examples of the aldehydes include formaldehyde, paraformaldehyde, trioxane, acetaldehyde, propionaldehyde, butyraldehyde, trimethylacetaldehyde, acrolein, crotonaldehyde, cyclohexanealdehyde, furfural, furylacrolein, benzaldehyde, terephthalaldehyde, phenylacetaldehyde, α-phenylpropylaldehyde, β-phenylpropylaldehyde, o-hydroxybenzaldehyde, m-hydroxybenzaldehyde, p-hydroxybenzaldehyde, o-methylbenzaldehyde, m-methylbenzaldehyde, p-methylbenzaldehyde, o-chlorobenzaldehyde, m-chlorobenzaldehyde, p-chlorobenzaldehyde, cinnamaldehyde, and the like. Among these, formaldehyde is preferred from the viewpoint of its availability. The aldehydes may be used alone or in a combination of two or more thereof.

Examples of the ketone-based solvent include methyl ethyl ketone, acetone, and the like. The ketone-based solvent may be used alone or in a combination of two or more thereof.

Examples of the acidic catalyst used under the acidic conditions include hydrochloric acid, sulfuric acid, formic acid, oxalic acid, paratoluenesulfonic acid, and the like. The acidic catalyst may be used alone or in a combination of two or more thereof.

The mass average molecular weight of the phenol formaldehyde resin is preferably 4,000 or more, preferably 7,000 or more, more preferably 9,000 or more, and particularly preferably 10,000 or more. Further, the mass average molecular weight of the phenol formaldehyde resin is usually 50,000 or less, preferably 40,000 or less, and more preferably 30,000 or less.

When the mass average molecular weight of the phenol formaldehyde resin is 4,000 or more, the obtained composition is less likely to exhibit low heat resistance and residual film ratio, and a favorable resolution tends to be achieved, and when the mass average molecular weight is 50,000 or less, the sensitivity of the obtained composition is less likely to be lowered.

### [Filler Component]

In the resist composition for pattern printing according to the present application, a filler (bulking agent) component is added to impart performances such as etchant resistance, concealability, and water resistance of a resist. Although any of organic fillers and inorganic fillers can be used as the filler component, an inorganic filler is preferred from the viewpoint of handleability and etchant resistance. Although not particularly limited, specific examples include: powders of magnesium carbonate, potassium titanate, calcium carbonate, calcium silicate, magnesium silicate, hydrous magnesium silicate, aluminum hydroxide, magnesium hydroxide, zinc borate, zinc molybdate, zinc hydroxide, mica, mica powder, beryllia, zirconia, zircon, fosterite, steatite, spinel, mullite, titania or the like; beads formed by spheronization of these powders, hollow beads, and glass fibers; and the like, and these may be used alone or in a combination of two or more thereof. In one embodiment of the resist composition for pattern printing according to the present application, it is preferable to use hydrous magnesium silicate as a filler component, although not particularly limited thereto. Here, the filler (bulking agent) is not particularly limited in terms of imparting performances such as etchant resistance, concealability and water resistance to the resist, but is desirably comprised of particles having a primary particle diameter thereof (or at least a major diameter for the case where the particles constituting the filler is not a spherical or substantially spherical in shape, and there is a difference between a major diameter and a minor diameter of the particles) of 1 μm or more, typically about 1 μm to 50 μm, and more preferably about 5 μm to 20 μm from the viewpoint of the function as a filler to impart etchant resistance, concealability, and water resistance as described above to the resist.

### [Thickener Component]

With regard to the resist composition for pattern printing according to the present application, thixotropic property is required for the pattern formation at the time of printing. Although the thickener for imparting the thixotropic property is not particularly limited, specific examples thereof include inorganic materials and organic materials as listed below.

Examples of the inorganic materials include talc, barium sulfate, barium titanate, silicas such as silicon oxide powders, finely powdered silicon oxide, amorphous silica, fused silica, crystalline silica and dry silica, clay, bentonite, and the like.

Examples of the organic materials include amide waxes, olefin waxes, castor oils, hydrogenated castor oils, and the like.

From the viewpoint of the balance between etchant resistance and thickening properties and the like, inorganic thickeners are preferred, and among them, fused silica, amorphous silica, dry silica, clay, and bentonite are more preferred. Further, hydrophilic fumed silica, which is a type of dry silica, is particularly preferred. Incidentally, when the thickener as exemplified hereinabove is in the form of a powder, the thickener is desirably, but is not particularly limited to, in the form of so-called nanoparticles having a primary particle diameter of less than 1 μm, typically about 20 nm to <1000 nm, and more preferably about 50 nm to 500 nm from the viewpoint of function as the thickener to impart the thixotropic property.

### [Polyfunctional (Meth)acrylate Component]

With regard to the resist composition for pattern printing according to the present application, in order to avoid deterioration of the peeling performance, it is important that, during the irradiation of the resist composition for pattern printing with an actinic radiation such as a UV exposure or irradiation with an electron beam after the development thereof into a predetermined pattern, an extremely thin film is formed on the surface of the resist pattern and the crosslinking is prevented from proceeding inside the resist pattern. Therefore, no polymerization initiator is added, and only the polyfunctional (meth)acrylate is added. The expression “(meth)acrylate” used herein means that both of “methacrylate” and “acrylate” are included as is well known.

The polyfunctional (meth)acrylate is not particularly limited, and specific examples thereof include, for example, dipentaerythritol hexa(meth)acrylate, dipentaerythritol penta(meth)acrylate, dipentaerythritol tetra(meth)acrylate, dipentaerythritol tri(meth)acrylate, pentaerythritol tetra(meth)acrylate, trimethylolpropane tri(meth)acrylate, neopentyl glycol di(meth)acrylate, ethylene glycol di(meth)acrylate, polyethylene glycol (n=2 to 15) di(meth)acrylate, polypropylene glycol (n=2 to 15) di(meth)acrylate, polybutylene glycol (n=2 to 15) di(meth)acrylate, 2,2-bis(4-(meth)acryloyloxyethoxyphenyl)propane, 2,2-bis(4-(meth)acryloyloxydiethoxyphenyl)propane, trimethylolpropane diacrylate, bis(2-(meth)acryloyloxyethyl)-hydroxyethyl-isocyanurate, trimethylolpropane tri(meth)acrylate, tris(2-(meth)acryloyloxyethyl) isocyanurate, pentaerythritol tri(meth)acrylate, and further, alkyl-modified (meth)acrylates, caprolactone-modified (meth)acrylates, ethylene oxide-modified (meth)acrylates or propylene oxide-modified (meth)acrylates of these polyfunctional (meth)acrylates, and (meth)acrylates of aliphatic polyols other than the aliphatic polyols described above. Furthermore, epoxy poly(meth)acrylates such as epoxy di(meth)acrylate obtainable by a reaction of bisphenol A-type diepoxide with (meth)acrylic acid; urethane poly(meth)acrylates such as urethane tri(meth)acrylate obtainable by a reaction of a trimer of 1,6-hexamethylene diisocyanate with 2-hydroxyethyl (meth)acrylate, urethane di(meth)acrylate obtainable by a reaction of isophorone diisocyanate with 2-hydroxypropyl (meth)acrylate, urethane hexa(meth)acrylate obtainable by a reaction of isophorone diisocyanate with pentaerythritol tri(meth)acrylate, urethane di(meth)acrylate obtainable by a reaction of dicyclomethane diisocyanate with 2-hydroxyethyl (meth)acrylate, and urethane di(meth)acrylate obtainable by a reaction of a urethanated reaction product of dicyclomethane diisocyanate and polytetramethylene glycol (n=6 to 15) with 2-hydroxyethyl (meth) acrylate; polyester poly(meth)acrylates such as polyester poly(meth)acrylates obtainable by a reaction of trimethylolethane with succinic acid and (meth)acrylic acid, and polyester poly(meth)acrylate obtainable by a reaction of trimethylolpropane with succinic acid, ethylene glycol and (meth)acrylic acid; and further, tris((meth)acryloxyethyl) isocyanurate, caprolactone-modified tris((meth)acryloxyethyl) isocyanurate, and the like. These polyfunctional (meth)acrylates may be used alone or in a combination of two or more thereof.

The number of (meth)acrylate functional groups possessed by such polyfunctional (meth)acrylate is not particularly limited, but the polyfunctional (meth)acrylate desirably has at least 3 or more, more preferably 3 to 6, and still more preferably 3 to 4 (meth)acrylate functional groups from the viewpoint of the reactivity upon the irradiation with the actinic radiation.

Among the polyfunctional (meth)acrylates listed above, dipentaerythritol hexa(meth)acrylate, dipentaerythritol penta(meth)acrylate, dipentaerythritol tetra(meth)acrylate, dipentaerythritol tri(meth)acrylate, pentaerythritol tetra(meth)acrylate, and trimethylolpropane tri(meth)acrylate are preferred from the viewpoint of balance between the reactivity upon an exposure to UV as the actinic radiation and the denseness of the film formed, and dipentaerythritol tetra(meth)acrylate, dipentaerythritol tri(meth)acrylate, pentaerythritol tetra(meth)acrylate, and trimethylolpropane tri(meth)acrylate are more preferred from the viewpoint of appropriate flexibility of the film.

The amount of the polyfunctional (meth)acrylate to be added is preferably 0.01 parts by mass or more and 10 parts by mass or less, and more preferably 0.1 parts by mass or more and 5 parts by mass or less with respect to 100 parts by mass of the resist composition for pattern printing, in terms of balance between surface curability and crack resistance of the film.

### [Photocatalyst]

In one preferred embodiment of the resist composition for pattern formation according to the present application, a photocatalyst component is blended into the resist composition for pattern formation, as described above. For example, because a photocatalyst component represented by titanium oxide has an ability to decompose an organic substance in the presence of light, the photocatalyst component is not usually used in a composition, such as a coating agent, and a resist agent, which contains an organic binder. In contrast, in the present application, a predetermined amount of photocatalyst is added into the resist material contrary to the usual practice based on the following viewpoint: when a resist pattern body is irradiated with an actinic radiation (UV light, in particular) after the printing of a predetermined resist pattern using a resist composition for pattern formation, a binder at a certain concentration is present in the resist pattern body, and thus the main performances including etchant resistance are not affected, and only a thin layer of the seepage portion is decomposed.

The photocatalytic component which can be used in the present application is not particularly limited as long as it has a photocatalytic activity to significantly decompose an organic substance in the thin layer of the seepage portion by irradiation with an actinic radiation such as UV light or an electron beam, in particular, UV light after pattern-printing. Any of known photocatalytic materials such as silver phosphate (Ag₃PO₄), titanium oxide (TiO₂), strontium titanate (SrTiO₃), cadmium selenide (CdSe), potassium tantalate (KTaO₃), cadmium sulfide (CdS), zirconium oxide (ZrO₂), niobium oxide (Nb₂O₅), zinc oxide (ZnO), iron oxide (Fe₂O₃), and tungsten oxide (WO₃) can be used, and among these materials, titanium oxide is preferable in light of the high photocatalytic activity thereof.

In the case of a photocatalytic substance comprised of an oxide semiconductor containing a plurality of metals, the photocatalytic substance can be formed by mixing and melting salts of the constituent elements. When removal of the solvent is necessary, firing and drying may be performed. Specifically, heating may be carried out at a predetermined temperature, e.g., 300° C. or higher, and preferably in an oxygen-containing atmosphere. This heating treatment allows the photocatalytic material to have a predetermined crystal structure. For example, in the titanium oxide (TiO₂), it has an anatase type or a mixed rutile-anatase type, and an anatase type is preferentially formed in the lower-temperature phase.

Further, it is also possible to use a material obtained by doping a transition metal (Pd, Pt, Cr, Ni, V, Mn, Fe, Ce, Mo, W or the like) into the photocatalyst material. The doping allows for an improvement of the photocatalytic activity or exertion of the photocatalytic activity by light of the visible light region (wavelength: 400 nm to 800 nm). The transition metal forms a new level within the forbidden band of the active photocatalyst with a wide band gap and can expand the absorption range of light up to the visible light region.

### [Titanium Oxide Component Having Photocatalytic Activity (Photocatalytic Titanium Oxide)]

As described above, a photocatalyst which is desirable as the photocatalyst to be blended in the preferred embodiment of the resist composition for pattern printing of the present application is titanium oxide having photocatalytic activity, and this titanium oxide having photocatalytic activity is desirably in the form of fine particles. The titanium dioxide used as a material of such fine particles may have a crystal structure of an anatase type or a rutile type. This is because the titanium dioxides differ in crystal structure, but the titanium dioxides have the same chemical property to be hydrated to form the hydroxyl group, and thus the surface modification thereof is possible. When high photocatalytic ability is desired, an anatase type may be appropriately selected, whereas when properties such as a high refractive index as in cosmetics are desired, a rutile type may be appropriately selected.

In addition, for the same reason, not only a single type of titanium oxide particles but also composite titanium dioxide particles comprised of titanium oxide and a magnetic material is suitably used. Further, from the viewpoint of the degree of freedom of the use form thereof, the primary particle diameter of the fine particles is 200 nm or less, preferably 1 nm or more and 200 nm or less, and desirably 2 nm or more and 200 nm or less. This is because the particle diameter of greater than 200 nm promotes an effect of gravity significantly acting on the fine particles, leading to a likelihood of the fine particles to settle down.

Further, the photocatalytic titanium oxide fine particles which can be used in preparing the resist composition for pattern printing according to the present application is desirably in the form of a dispersion in which the photocatalytic titanium oxide fine particles are dispersed in an aqueous or alcohol-based solvent. Further, it is preferable to treat the particle surface with an organic functional group in order to achieve favorable dispersibility in such a dispersion. For example, when the surface of the particle is treated with an amine, the particles are stably present over a long period of time due to an electric repulsive force acting between the particles without aggregation. In addition, the particles are extremely stable against pH variation and addition of inorganic salts.

The amount of the photocatalyst component, titanium oxide having photocatalytic activity in particular, which can be blended in the preferred embodiment of the resist composition for pattern printing of the present application, is not particularly limited, but is preferably 0.1 parts by mass or more and 20 parts by mass or less, and more preferably 0.5 parts by mass or more and 10 parts by mass or less with respect to 100 parts by mass of the resist composition for pattern printing. When the blending amount is within such a range, favorable degradability is exhibited substantially only at the seepage portion from the end of the pattern after the printing of the resist pattern, and removal of the seepage portion can be facilitated.

### <Diluent Solvent>

The diluent solvent is not particularly limited as long as the dispersibility of the resist composition for pattern printing of the present application is favorable, and it dries quickly by heating without excessively drying at the time of printing and does not cause defects such as bubbles in the resist pattern.

Specific examples include cycloalkyl alcohol, cycloalkyl acetate, alkylene glycol, alkylene glycol diacetate, alkylene glycol monoether, alkylene glycol dialkyl ether, alkylene glycol monoether acetate, dialkylene glycol monoether, dialkylene glycol dialkyl ether, dialkylene glycol monoalkyl ether acetate, trialkylene glycol monoether, trialkylene glycol monoether acetate, 3-methoxybutanol, 3-methoxybutanol acetate, tetrahydrofurfuryl alcohol, tetrahydrofurfuryl alcohol acetate, terpene-based compounds and derivatives thereof, and the like.

Examples of the cycloalkyl alcohol include cycloalkyl alcohols having 3 to 15 ring atoms and optionally being substituted with a C₁₋₅ alkyl group and the like, such as cyclopentanol, cyclohexanol, cyclooctyl alcohol, methylcyclohexyl alcohol, ethylcyclohexyl alcohol, propylcyclohexyl alcohol, i-propylcyclohexyl alcohol, butylcyclohexyl alcohol, i-butylcyclohexyl alcohol, s-butylcyclohexyl alcohol, t-butylcyclohexyl alcohol, and pentylcyclohexyl alcohol, and the like.

Examples of the cycloalkyl acetate include cycloalkyl acetates having 3 to 15 ring atoms and optionally being substituted with a C₁₋₅ alkyl group and the like, such as cyclohexyl acetate, cyclopentyl acetate, cyclooctyl acetate, methylcyclohexyl acetate, ethylcyclohexyl acetate, propylcyclohexyl acetate, i-propylcyclohexyl acetate, butylcyclohexyl acetate, i-butylcyclohexyl acetate, s-butylcyclohexyl acetate, t-butylcyclohexyl acetate, and pentylcyclohexyl acetate, and the like.

Examples of the alkylene glycol include ethylene glycol, propylene glycol, 1,3-propanediol, 1,3-butylene glycol, 1,4-butanediol, 1,5-pentanediol, 1,6-hexanediol, and the like.

Examples of the alkylene glycol diacetate include ethylene glycol diacetate, propylene glycol diacetate, 1,3-propanediol diacetate, 1,3-butylene glycol diacetate, 1,4-butanediol diacetate, 1,5-pentanediol diacetate, 1,6-hexanediol diacetate, and the like.

Examples of the alkylene glycol monoether include: ethylene glycol mono C₁₋₅ alkyl ethers such as ethylene glycol monomethyl ether, ethylene glycol monoethyl ether, ethylene glycol monopropyl ether, ethylene glycol monobutyl ether, and ethylene glycol monopentyl ether; propylene glycol mono C₁₋₅ alkyl ethers such as propylene glycol monomethyl ether, propylene glycol monoethyl ether, propylene glycol monopropyl ether, propylene glycol monobutyl ether, and propylene glycol monopentyl ether, and the like.

Examples of the alkylene glycol dialkyl ether include: ethylene glycol C₁₋₅ alkyl (linear or branched) C₁₋₅ alkyl (linear or branched) ethers (terminal alkyl groups being symmetric) such as ethylene glycol dimethyl ether, ethylene glycol diethyl ether, ethylene glycol dipropyl ether, ethylene glycol dibutyl ether, and ethylene glycol dipentyl ether; ethylene glycol C₁₋₅ alkyl (linear or branched) C₁₋₅ alkyl (linear or branched) ethers (terminal alkyl groups being asymmetric) such as ethylene glycol ethyl methyl ether, ethylene glycol methyl propyl ether, ethylene glycol butyl methyl ether, ethylene glycol methyl pentyl ether, ethylene glycol ethyl propyl ether, ethylene glycol butyl ethyl ether, ethylene glycol ethyl pentyl ether, ethylene glycol butyl propyl ether, ethylene glycol propyl pentyl ether, and ethylene glycol butyl pentyl ether; propylene glycol C₁₋₅ alkyl (linear or branched) C₁₋₅ alkyl (linear or branched) ethers (terminal alkyl groups being symmetric) such as propylene glycol dimethyl ether, propylene glycol diethyl ether, propylene glycol dipropyl ether, propylene glycol dibutyl ether, and propylene glycol dipentyl ether; propylene glycol C₁₋₅ alkyl (linear or branched) C₁₋₅ alkyl (linear or branched) ethers (terminal alkyl groups being asymmetric) such as propylene glycol ethyl methyl ether, propylene glycol methyl propyl ether, propylene glycol butyl methyl ether, propylene glycol methyl pentyl ether, propylene glycol ethyl propyl ether, propylene glycol butyl ethyl ether, propylene glycol ethyl pentyl ether, propylene glycol butyl propyl ether, propylene glycol propyl pentyl ether, and propylene glycol butyl pentyl ether; and the like.

Examples of the alkylene glycol monoalkyl ether acetate include: ethylene glycol mono C₁₋₅ alkyl ether acetates such as ethylene glycol monomethyl ether acetate, ethylene glycol monoethyl ether acetate, ethylene glycol monopropyl ether acetate, ethylene glycol monobutyl ether acetate, and ethylene glycol monopentyl ether acetate; propylene glycol mono C₁₋₅ alkyl ether acetates such as propylene glycol monoethyl ether acetate, propylene glycol monopropyl ether acetate, propylene glycol monobutyl ether acetate, and propylene glycol monopentyl ether acetate; and the like (including isomers thereof).

Examples of the dialkylene glycol monoether include: diethylene glycol mono C₁₋₅ alkyl ethers such as diethylene glycol monomethyl ether, diethylene glycol monoethyl ether, diethylene glycol monopropyl ether, diethylene glycol monobutyl ether, and diethylene glycol monopentyl ether; dipropylene glycol mono C₁₋₅ alkyl ethers such as dipropylene glycol monomethyl ether, dipropylene glycol monoethyl ether, dipropylene glycol monopropyl ether, dipropylene glycol monobutyl ether, and dipropylene glycol monopentyl ether; and the like (including isomers thereof).

Examples of the dialkylene glycol dialkyl ether include: diethylene glycol C₁₋₅ alkyl (linear or branched) C₁₋₅ alkyl (linear or branched) ethers (terminal alkyl groups being symmetric) such as diethylene glycol dimethyl ether, diethylene glycol diethyl ether, diethylene glycol dipropyl ether, diethylene glycol dibutyl ether, and diethylene glycol dipentyl ether; diethylene glycol C₁₋₅ alkyl (linear or branched) C₁₋₅ alkyl (linear or branched) ethers (terminal alkyl groups being asymmetric) such as diethylene glycol ethyl methyl ether, diethylene glycol methyl propyl ether, diethylene glycol butyl methyl ether, diethylene glycol methyl pentyl ether, diethylene glycol ethyl propyl ether, diethylene glycol butyl ethyl ether, diethylene glycol ethyl pentyl ether, diethylene glycol butyl propyl ether, diethylene glycol propyl pentyl ether, and diethylene glycol butyl pentyl ether; dipropylene glycol C₁₋₅ alkyl (linear or branched) C₁₋₅ alkyl (linear or branched) ethers (terminal alkyl groups being symmetric) such as dipropylene glycol dimethyl ether, dipropylene glycol diethyl ether, dipropylene glycol dipropyl ether, dipropylene glycol dibutyl ether, and dipropylene glycol dipentyl ether; dipropylene glycol C₁₋₅ alkyl (linear or branched) C₁₋₅ alkyl (linear or branched) ethers (terminal alkyl groups being asymmetric) such as dipropylene glycol ethyl methyl ether, dipropylene glycol methyl propyl ether, dipropylene glycol butyl methyl ether, dipropylene glycol methyl pentyl ether, dipropylene glycol ethyl propyl ether, propylene glycol butyl ethyl ether, dipropylene glycol ethyl pentyl ether, dipropylene glycol butyl propyl ether, dipropylene glycol butyl propyl ether, and dipropylene glycol propyl pentyl ether; and the like (including isomers thereof).

Examples of the dialkylene glycol monoalkyl ether acetate include: diethylene glycol mono C₁₋₅ alkyl ether acetates such as diethylene glycol monomethyl ether acetate, diethylene glycol monopropyl ether acetate, diethylene glycol monobutyl ether acetate, and diethylene glycol monopentyl ether acetate; dipropylene glycol mono C₁₋₅ alkyl ether acetates such as dipropylene glycol monoethyl ether acetate, dipropylene glycol monopropyl ether acetate, dipropylene glycol monobutyl ether acetate, and dipropylene glycol monopentyl ether acetate; and the like (including isomers thereof).

Examples of the trialkylene glycol monoether include: triethylene glycol mono C₁₋₅ alkyl ethers such as triethylene glycol monomethyl ether, triethylene glycol monoethyl ether, triethylene glycol monopropyl ether, triethylene glycol monobutyl ether, and triethylene glycol monopentyl ether; tripropylene glycol mono C₁₋₅ alkyl ethers such as tripropylene glycol monomethyl ether, tripropylene glycol monoethyl ether, tripropylene glycol monopropyl ether, tripropylene glycol monobutyl ether, and tripropylene glycol monopentyl ether; and the like (including isomers thereof).

Examples of the trialkylene glycol monoether acetate include: triethylene glycol mono C₁₋₅ alkyl ether acetates such as triethylene glycol monomethyl ether acetate, triethylene glycol monoethyl ether acetate, triethylene glycol monopropyl ether acetate, triethylene glycol monobutyl ether acetate, and triethylene glycol monopentyl ether acetate; tripropylene glycol mono C₁₋₅ alkyl ether acetates such as tripropylene glycol monomethyl ether acetate, tripropylene glycol monoethyl ether acetate, tripropylene glycol monopropyl ether acetate, tripropylene glycol monobutyl ether acetate, and tripropylene glycol monopentyl ether acetate; and the like (including isomers thereof).

Examples of the terpene-based compounds and derivatives thereof include terpineol, terpineol acetate, dihydroterpineol, dihydroterpinyl acetate, dihydroterpinyl propionate, limonene, menthane, menthol, and the like.

The amount of the diluent to be added in the resist composition for pattern printing according to the present application is preferably 0.1 parts by mass or more and 30 parts by mass or less, more preferably 1 part by mass or more and 20 parts by mass or less with respect to 100 parts by mass of the resist composition for pattern printing, in light of balance between surface curability and crack resistance of the film. In addition, in the embodiment in which a photocatalyst component such as titanium oxide having photocatalytic activity is blended into the resist composition for pattern printing, in the case where the photocatalytic titanium oxide fine particles are in the form of a dispersion in which the photocatalytic titanium oxide fine particles are dispersed in an aqueous or alcohol-based solvent as described above, the dispersion medium of the dispersion can be used as a diluent or a part of a diluent in the resist composition for pattern printing.

### <Leveling Agent>

A leveling agent may be added to the resist composition for pattern printing in order to adjust the surface irregularities when cured. Alternatively, the leveling agent may be added to ensure smoothness of the coating film and prevent breakage when the resist composition for pattern printing is coated on an adherend.

Generally, examples of the leveling agent include fluorine-based leveling agents, silicone-based leveling agents, acrylic leveling agents, ether-based leveling agents, and ester-based leveling agents, and any of these leveling agents may be used in the resist composition for pattern printing according to the present application.

### <Antifoaming Agent>

An antifoaming agent may be added to the resist composition for pattern printing for the purpose of preventing generation of bubbles which may be generated in screen printing or the like. Preferred examples include acrylic antifoaming agents, silicon-based antifoaming agents, and fluorine-based antifoaming agents.

### <Adhesiveness Imparting Agent>

An adhesiveness imparting agent may be added to the resist composition for pattern printing, in order to improve adhesion to the base material.

As the adhesiveness imparting agent, a crosslinkable silyl group-containing compound and a vinyl monomer having a polar group are preferred, and further, a silane coupling agent and an acidic group-containing vinyl monomer are preferred.

As the silane coupling agent, for example, a silane coupling agent having both a functional group having, in the molecule thereof, an atom other than a carbon atom and a hydrogen atom, such as an epoxy group, an isocyanate group, an isocyanurate group, a carbamate group, an amino group, a mercapto group, a carboxyl group, a halogen group and a (meth)acryl group, and a cross-linkable silyl group may be used.

### <Plasticizer>

A plasticizer may be added to the resist composition for pattern printing in order to adjust viscosity, slump property, or mechanical properties such as hardness, tensile strength, or elongation when cured.

Examples of the plasticizer include, but are not particularly limited to:

phthalic acid ester compounds such as dibutyl phthalate, diisononyl phthalate (DINP), diheptyl phthalate, di(2-ethylhexyl) phthalate, diisodecyl phthalate (DIDP), and butyl benzyl phthalate; terephthalic acid ester compounds such as bis(2-ethylhexyl) 1,4-benzenedicarboxylate, for example, EASTMAN 168 (manufactured by EASTMAN CHEMICAL);
non-phthalic acid ester compounds such as 1,2-cyclohexanedicarboxylic acid diisononyl ester, for example, Hexamoll DINCH (manufactured by BASF);
aliphatic polyhydric carboxylic acid ester compounds such as dioctyl adipate, dioctyl sebacate, dibutyl sebacate, diisodecyl succinate, and tributyl acetylcitrate;
unsaturated fatty acid ester compounds such as butyl oleate, and methyl acetylricinoleate;
alkylsulfonic acid phenyl esters, for example, Mesamoll (manufactured by LANXESS);
phosphoric acid ester compounds such as tricresyl phosphate, and tributyl phosphate;
trimellitic acid ester compounds;
chlorinated paraffins;
hydrocarbon-based oils such as alkyldiphenyl, and partially hydrogenated terphenyl;
process oils;
epoxy plasticizers such as epoxidized soybean oils, and epoxy benzyl stearate; and the like.

### [Method for Producing Resist Composition for Pattern Printing]

The resist composition for pattern printing according to the present application can be generally produced by mixing and stirring the binder, the polyfunctional (meth)acrylate, the thickener, the filler, and the photocatalyst component such as the photocatalytic titanium oxide in this order. At this production, after the binder, the polyfunctional (meth)acrylate, and the thickener are added, the mixture is sufficiently stirred, and kneaded once, and further, the filler and the photocatalytic titanium oxide are added, mixed, and finally kneaded, whereby a resist composition for pattern printing having a thixotropic property imparted thereto can be effectively obtained.

Examples of the apparatus used for kneading include a stirring and defoaming apparatus such as a three-roll mill, a planetary mixer, and a planetary stirring and defoaming apparatus. The apparatus can be used in a combination of a plurality of types.

Incidentally, when kneading, attention should be paid such that volatilization of the solvent component due to heat generation during kneading is avoided. The temperature during the kneading is preferably 0° C. or higher and 80° C. or lower, more preferably 5° C. or higher and 60° C. or lower, and still more preferably 10° C. or higher and 50° C. or lower from the viewpoint of the balance between addition of the thickener and the kneading of the filler added.

Further, it is also possible to obtain the resist composition for pattern printing according to the present application by mixing and kneading the polyfunctional (meth)acrylate, and the photocatalyst component such as the photocatalytic titanium oxide into a resist composition for pattern printing in which the binder, the thickener, and the filler are mixed beforehand. In this case, the kneading method is the same as described above.

### [Properties of Present Resist Composition for Pattern Printing]

The resist composition for pattern printing according to the present application can be used for etching and patterning, for example, a conductive layer of a device such as an electrode film, an electrode substrate or a solar battery, as described above. For example, in the case of FPD, the resist composition for pattern printing can be used for circuit-forming (etching) of an ITO (Indium Tin Oxide) film and FPC (Flexible Printed Circuits) made of polyimide. Further, in the case of a solar battery, the resist composition for pattern printing according to the present application can be used to form a photoelectron generating and transferring layer beneath a collecting electrode on a silicon substrate, and an amorphous layer pattern only on one main surface of a silicon substrate, in particular, in a back electrode type solar battery.

### [Method for Producing Solar Battery Cell]

A method for producing a solar battery cell according to the present application includes: a printing step of printing a resist pattern on a substrate having a semiconductor layer using the resist composition for pattern printing according to the present application, particularly desirably using the resist composition for pattern printing according to a preferred embodiment of the present application containing the photocatalyst component in addition to the binder component, the filler component, the thickener component, and the polyfunctional (meth)acrylate, but not containing the photoinitiator, a step of irradiating the resist pattern with an actinic radiation, a step of drying the resist pattern, an etching step of etching a part of the semiconductor layer and a part of the resist pattern, and a peeling step of peeling off the residual resist pattern.

According to the method for producing a solar battery cell according to the present application including such steps, the seepage from the resist pattern can be reduced, the seepage portion can be decomposed by simple irradiation with the actinic radiation, and thus excellent etchability of the target region can be maintained.

Further, in the method for producing a solar battery cell according to the present application, at least the resist pattern is irradiated with an actinic radiation such as UV light or an electron beam after the printing step prior to the etching step or in the etching step. The irradiation with the actinic radiation causes a thin film to be formed only on the surface of the resist layer developed into a pattern through oxidative crosslinking of the polyfunctional (meth)acrylate component blended in the resist composition for pattern printing according to the present application, to thereby suppress the flow of the resist from the predetermined pattern and prevent the seepage.

In addition, in the method for producing a solar battery cell according to the present application, in the etching step, one or more selected from the group consisting of an aqueous acid solution, an aqueous alkaline solution and water are brought into contact with the semiconductor layer and/or the resist pattern during or after the irradiation with the actinic radiation. This process allows for easy removal of the slightly seeping portion at the end of the resist pattern. The aqueous acid solution, the aqueous alkaline solution, or the water to be brought into contact also depends on the type of the binder component used in the resist composition for pattern printing according to the present application, but as an example, the pH of the aqueous alkaline solution may be about 8.0 to 13.5, and the pH of the aqueous acid solution may be about 1.0 to 6.0. Further, as the water, distilled water, deionized water, ultrafiltered water, pure water, ultrapure water, or the like can be used, but preferably water classified as pure water or ultrapure water is used. Incidentally, the pure water described herein is not particularly limited, and is water that can be obtained, for example, through a process including: a deoxygenation step of passing water through a reverse osmosis membrane after heating, degassing water in a degassing device such as a vacuum tank, or aerating water with a high-purity nitrogen gas, to thereby reduce the dissolved oxygen to about 0.1 mg/L, then cooling water with a heat exchanger, and oxidizing and decomposing a trace amount of an organic substance by an ultraviolet radiation or ozone, then passing this through a highly purified and washed ion exchange resin to remove the ionized organic substance, and further passing this through an ultrafiltration membrane of a hollow fiber membrane to capture organic substances which cannot be reduced in molecular weight by the ultraviolet radiation as well as fine particles and live bacteria.

In addition, in the method for producing a solar battery cell according to the present application, in the peeling step, an aqueous alkaline solution or an alcohol-containing aqueous solution is brought into contact with the semiconductor layer and/or the resist pattern, although not particularly limited. Although depending on the type of the binder component used in the resist composition for pattern printing according to the present application, the contact with such a solution enables the resist layer remaining after the etching treatment to be easily peeled off and removed. The pH of the aqueous alkaline solution may be 8.0 to 13.0, or 8.5 to 12.0. Examples of the alcohol in the alcohol-containing aqueous solution include isopropanol, n-butanol, 2-butanol, isobutanol, hexanol, cyclohexanol, n-octanol, 2-ethyl-hexyl alcohol, n-decanol, 1-(2-methoxy-2-methoxyethoxy)-2-propanol, 3-methoxy-3-methyl butanol, 1-methoxy-2-propanol, 1-ethoxy-2-propanol, benzyl alcohol, and the like.

### [Solar Battery]

The solar battery according to the present application includes a circuit pattern obtained using a resist composition for pattern printing according to the present application or the method for producing a circuit pattern according to the present application. As described above, because a residue is less likely to remain on this circuit pattern, a solar battery electrode having high electrode performances is likely to be obtained from this circuit pattern. As a result, the solar battery according to the present application is likely to exhibit high conversion efficiency.

The present application is not limited to the above-described embodiments, and various modifications can be made within the scope defined in the claims. In other words, embodiments obtained by combining technical means appropriately modified within the scope defined in the claims are also included in the technical scope of the present application.

As described above, the resist composition for pattern printing according to the present application is a resist composition for pattern printing containing the binding agent, the filler component, the thickener component, the polyfunctional (meth)acrylate component, and not containing the photoinitiator, and in a preferred embodiment, the resist composition for pattern printing further containing a photocatalyst component, and the properties described hereinabove and in the following Examples can be each analyzed by the following measurement methods.

### <Performance Evaluation of Resist Composition for Pattern Printing>

The performance of the resist composition for pattern printing according to the present application is evaluated by the following methods.

### (Viscosity)

Viscosity is measured using a B-type viscometer as described in JIS Z 8803. Especially, because the resist composition according to the present application is highly viscous, the viscosity can be measured with HB type (manufactured by EKO Instruments Co. Ltd.). The viscosity measured at 25±1° C. using a spindle SC4-14 as a rotor is defined as the viscosity specified herein.

### (Printability)

A predetermined pattern is printed on a silicon wafer on which an amorphous layer was formed, using a screen printing plate with an L/S of 400/600 μm and an emulsion thickness of 20 μm and a screen printing machine at a squeegee pressure of 0.20 MPa and a speed of 100 ram/min, and the printability was examined in terms of blurring, the spread of the pattern, and the thickness of the pattern. The evaluations are made based on the following evaluation criteria.

A: No blurring is observed. The pattern spread width is 50 μm or less, the thickness is ±3 μm or less with respect to the setting, and the screen printing is possible, and no problem in the printability is caused.
B: No blurring is observed. The pattern spread width is greater than 50 μm and 70 μm or less, the thickness is ±5 μm or less with respect to the setting, and a slight problem is caused in screen printing.
C: Blurring is observed. The pattern spread width is greater than 70 μm. The thickness is ±5 μm or more with respect to the setting, and the screen printing is unfavorable.
(State of Seepage from End of Resist Pattern)

A resist with a line width of 450 μm, an interline space of 100 μm, and a thickness of 40 μm is screen-printed on a cell in which a p-layer (hole transport layer) is formed on a textured silicon wafer, and dried at 120° C. for 30 minutes. Then, the entire surface of the pattern is exposed to UV (integrated light amount of 1,830 mJ/cm², exposure device: Light Hammer L-6 (manufactured by Heraeus)), and then is immersed in pure water for 5 minutes. For some of the samples, an end of the resist pattern is observed after the UV exposure before immersion in pure water with an optical microscope, and the width of a transparent film formed by seepage of a binder component dissolved in a solvent (a seepage width after UV exposure) is measured. Further, the rest of the samples are removed from pure water after a lapse of a predetermined time of immersion, and after natural drying, the end of the resist pattern is observed with an optical microscope, and the width of the transparent film formed by the seepage of the binder component dissolved in the solvent, or a seepage width after the UV exposure and water immersion treatment is measured.

### (Ozone and Hydrofluoric Acid Resistance)

A resist with a line width of 450 μm, an interline space of 100 μm, and a thickness of 40 μm is screen-printed on a cell in which a p-layer (hole transport layer) is formed on a textured silicon wafer, and dried at 120° C. for 30 minutes, to form a resist pattern. The cell is immersed in an etching tank containing 40 ppm of ozone, and 7% by mass of hydrofluoric acid for 10 minutes, then the state of the p-layer was observed with a scanning electron microscope (SEM), and the ozone and hydrofluoric acid resistance is evaluated according to the following evaluation criteria.

Favorable: the line width of the p-layer is 400 μm or more and 450 μm or less;
Acceptable: the line width of the p-layer is 350 μm or more and less than 400 μm; and
Poor: the line width of the p-layer is less than 350 μm.

### (Resist Peelability)

The cell subjected to the ozone and hydrofluoric acid treatment described above is immersed in an aqueous solution in which 0.4% by mass of potassium hydroxide and 1.0% by mass of borax are dissolved, with shaking (shaking stroke=3 cm, speed=60 rpm) for 2 minutes, and the state of resist peeling is observed with an optical microscope.

### EXAMPLES

Hereinafter, the present application will be specifically described by way of Examples, but the present application is not limited by these Examples.

### <Composition of Resist Composition for Pattern Printing (See Table 1)>

### Example 1

Into a vessel dedicated to blending (09×08×94 mm, internal volume of 470 cc, made of polypropylene) were added 64 g of PHORET (trademark) ZAH-110 (an acrylic binder manufactured by Soken Chemical & Engineering Co., Ltd., a nonvolatile component=35% by mass, amount of a carboxylic acid functional group=96 mgKOH/g, solvent component=propylene glycol monomethyl ether acetate, viscosity=300 mPa·s) as a binder component, 3 g of LIGHT ACRYLATE TMP-A (trimethylolpropane triacrylate, manufactured by Kyoeisha Chemical Co., Ltd.) as a polyfunctional (meth)acrylate, and 1 g of AEROSIL #300 (hydrophilic fumed silica, manufactured by Evonik Japan Co., Ltd.) as a thickener. After manually stirring with a medicine spoon, the mixture was subjected to shearing and stirring using a dedicated stirring and defoaming apparatus (product number: Thinky mixer ARV-310, manufactured by Thinky Corporation). Then, 15 g of hydrous magnesium silicate (manufactured by Wako Pure Chemical Industries, Ltd.) and 17 g of TKD-701 (manufactured by Tayca Corporation, anatase type crystals, primary particle diameter=6 nm, an IPA slurry with solid content of 17%) as photocatalytic titanium oxide were added, and mixing, stirring and defoaming were carried out as described above, to obtain a resist composition for pattern printing. The properties including the viscosity, the printability, the state of seepage from the end of the resist pattern, the ozone and hydrofluoric acid resistance and the resist peelability were evaluated on the resist composition for pattern printing obtained thus according to the above-described measurement methods. The results obtained are shown in Table 1.

### Example 2

A resist composition for pattern-printing was obtained in the same manner as in Example 1, except that the photocatalytic titanium oxide in Example 1 was changed to TKD-702 (manufactured by Tayca Corporation, anatase-type crystals, primary particle size=6 nm, an IPA slurry with solid content of 16%), and the same characterization was performed. The results obtained are shown in Table 1.

### Example 3

A resist composition for pattern printing was obtained in the same manner as in Example 1, except that the photocatalytic titanium oxide in Example 1 was changed to 10 g of STS-01 (manufactured by Ishihara Sangyo Kaisha, Ltd., anatase type crystals, primary particle diameter=7 nm, a water-based sol with solid content of 30%) and the amount of the hydrous magnesium silicate was changed to 22 g, and the same characterization was performed. The results obtained are shown in Table 1.

### Example 4

A resist composition for pattern printing was obtained in the same manner as in Example 1, except that the photocatalytic titanium oxide in Example 1 was changed to 10 g of STS-02 (manufactured by Ishihara Sangyo Kaisha, Ltd., anatase type crystals, primary particle diameter=7 nm, a water-based sol with solid content of 30%) and the amount of the hydrous magnesium silicate was changed to 22 g, and the same characterization was performed. The results obtained are shown in Table 1.

### Example 5

A resist composition for pattern printing was obtained in the same manner as in Example 1, except that TKD-701 in Example 1 was changed to STS-01, and the same characterization was performed. The results obtained are shown in Table 1.

### Example 6

A resist composition for pattern printing was obtained in the same manner as in Example 5, except that STS-01 in Example 5 was changed to STS-02, and the same characterization was performed. The results obtained are shown in Table 1.

### Example 7

A resist composition for pattern printing was obtained in the same manner as in Example 1, except that 3 g of PE-3A (manufactured by Kyoeisha Chemical Co., Ltd., pentaerythritol triacrylate) was added instead of the LIGHT ACRYLATE TMP-A in Example 1, and the same characterization was performed. The results obtained are shown in Table 1.

### Example 8

A resist composition for pattern printing was obtained in the same manner as in Example 3, except that 3 g of PE-3A (manufactured by Kyoeisha Chemical Co., Ltd., pentaerythritol triacrylate) was added instead of LIGHT ACRYLATE TMP-A in Example 3, and the same characterization was performed. The results obtained are shown in Table 1.

### Example 9

A resist composition for pattern printing was obtained in the same manner as in Example 1, except that TKD-701 in Example 1 was not added and the amount of ZAH-100 was changed to 81 g, and the same characterization was performed. The results obtained are shown in Table 1.

### Comparative Example 1

A resist composition for pattern printing was obtained in the same manner as in Example 1, except that TMP-A and TKD-701 in Example 1 were not added, the amount of ZAH-110 was changed to 74 g, and the amount of the hydrous magnesium silicate was changed to 22 g, and the same characterization was performed. The results obtained are shown in Table 1.

### Comparative Example 2

A resist composition for pattern printing was obtained in the same manner as in Example 1, except that the amount of ZAH-110 was changed to 63.8 g, and 0.2 g of IRGACURE TPO (manufactured by BASF Japan Ltd., 2,4,6-trimethylbenzoyl-diphenyl-phosphine oxide) was added, and the same characterization was performed. The results obtained are shown in Table 1.

### (Printability)

The resist compositions for pattern printing of Examples 1 to 8 and Comparative Examples 1 and 2 exhibited favorable screen printing.

### (Seepage-Preventive Property)

The width of the transparent thin film at the end of the resist was 200 μm in Comparative Example 1. On the other hand, the width of the transparent thin film at the end of the resist was 150 μm or less in Examples 1 to 8 even after pattern printing, demonstrating a favorable effect of suppressing the seepage, and further, the transparent thin film could be removed by water immersion to a more favorable extent indicated by a width of 50 μm or less, demonstrating a favorable effect of suppressing the seepage.

### (Etchant Resistance)

The etchant resistance to 5.5% hydrofluoric acid/40 ppm, which is an etchant for amorphous of a solar battery cell or the like, was not different between Comparative Examples 1 and 2 and Examples 1 to 8, and very favorable results were obtained.

### (Resist Peelability)

With regard to the peelability evaluation of the resist pattern with the aqueous alkaline solution of a 4% KOH+8% KBO₃, in Examples 1 to 8 and Comparative Example 1, the peeling of the resist was completed in 10 minutes of immersion, indicating favorable results, whereas in Comparative Example 2 in which the photoinitiator was added, it was found that curing of the resist pattern ensued, and no change in shape of the resist pattern was found even when the resist pattern was immersed in the peeling solution for 10 minutes, indicating the lowered peelability.

TABLE 1

| Regarding materials | | | | | | | | |
|---|---|---|---|---|---|---|---|---|
| Class | Grade name | Maker | Example 1 | Example 2 | Example 3 | Example 4 | Example 5 | Example 6 |
| Binder | ZAH-110 | Soken Chemical & | 64 | 64 | 64 | 64 | 64 | 64 |
| Component | | Engineering | | | | | | |
| Thickener | AEROSIL#300 | Evonik Japan | 1 | 1 | 1 | 1 | 1 | 1 |
| Filler | Hydrous | Wako Pure | 15 | 15 | 22 | 22 | 15 | 15 |
| | magnesium | Chemical | | | | | | |
| | silicate | Industries | | | | | | |
| Polyfunctional | TMPA | Kyoeisha | 3 | 3 | 3 | 3 | 3 | 3 |
| acrylate | | Chemical | | | | | | |
| | PE-3A | Kyoeisha | | | | | | |
| | | Chemical | | | | | | |
| Photocatalytic | TKD-701 | Tayca | 17 | | | | | |
| Titanium Oxide | (IPA 17% slurry) | Corporation | | | | | | |
| | TKD-702 | Tayca | | 17 | | | | |
| | (IPA 16% slurry) | Corporation | | | | | | |
| | STS-01(30% | Ishihara Sangyo | | | 10 | | 17 | |
| | aqueous slurry) | Kaisha | | | | | | |
| | STS-02(30% | Ishihara Sangyo | | | | 10 | | 17 |
| | aqueous slurry) | Kaisha | | | | | | |
| Photoinitiator | IRGACURE_TPO | BASF Japan | | | | | | |
| Evaluation | Viscosity(Pa · s) | B-type | 55 | 58 | 65 | 66 | 75 | 77 |
| results | | viscometer | | | | | | |
| | Printability | Observation | A | A | A | A | A | A |
| | | with | | | | | | |
| | | microscope | | | | | | |
| | Seepage width | μm | ≈100 | ≈100 | ≈100 | ≈50 | ≈50 | ≈50 |
| | after UV | | | | | | | |
| | exposure | | | | | | | |
| | Seepage width | μm | ≈50 | ≤50 | ≤20 | ≈50 | ≤20 | ≈0 |
| | after UV | | | | | | | |
| | exposure and | | | | | | | |
| | water immersion | | | | | | | |
| | Ozone and | | Favorable | Favorable | Favorable | Favorable | Favorable | Favorable |
| | Hydrofluoric | | | | | | | |
| | Acid Resistance | | | | | | | |
| | Alkali peeling | Visual | Complete | Complete | Complete | Complete | Complete | Complete |
| | after UV | inspection | peeling | peeling | peeling | peeling | peeling | peeling |
| | exposure | | in 10 min | in 10 min | in 10 min | in 10 min | in 10 min | in 10 min |

| Regarding materials | | | | | | Comparative | Comparative |
|---|---|---|---|---|---|---|---|
| Class | Grade name | Maker | Example 7 | Example 8 | Example 9 | Example 1 | Example 2 |
| Binder | ZAH-110 | Soken Chemical & Engineering | 64 | 64 | 81 | 74 | 63.8 |
| Component | | | | | | | |
| Thickener | AEROSIL#300 | Evonik Japan | 1 | 1 | 1 | 1 | 1 |
| Filler | Hydrous | Wako Pure Chemical | 15 | 22 | 15 | 22 | 15 |
| | magnesium | Industries | | | | | |
| | silicate | | | | | | |
| Polyfunctional | TMPA | Kyoeisha Chemical | | | 3 | | 3 |
| acrylate | PE-3A | Kyoeisha Chemical | 3 | 3 | | | |
| Photocatalytic | TKD-701 | Tayca Corporation | 17 | | | | 17 |
| Titanium Oxide | (IPA 17% slurry) | | | | | | |
| | TKD-702 | Tayca Corporation | | | | | |
| | (IPA 16% slurry) | | | | | | |
| | STS-01(30% | Ishihara Sangyo Kaisha | | 10 | | | |
| | aqueous slurry) | | | | | | |
| | STS-02(30% | Ishihara Sangyo Kaisha | | | | | |
| | aqueous slurry) | | | | | | |
| Photoinitiator | IRGACURE_TPO | BASF Japan | | | | | 0.2 |
| Evaluation | Viscosity(Pa · s) | B-type viscometer | 55 | 65 | 20 | 38 | 55 |
| results | Printability | Observation with | A | A | A | A | A |
| | | microscope | | | | | |
| | Seepage width | μm | ≈50 | ≈50 | ≈150 | ≈200 | ≈100 |
| | after UV | | | | | | |
| | exposure | | | | | | |
| | Seepage width | μm | ≈0 | ≤20 | ≈150 | ≈200 | ≈50 |
| | after UV | | | | | | |
| | exposure and | | | | | | |
| | water immersion | | | | | | |
| | Ozone and | | Favorable | Favorable | Favorable | Favorable | Favorable |
| | Hydrofluoric | | | | | | |
| | Acid Resistance | | | | | | |
| | Alkali peeling | Visual inspection | Complete | Complete | Complete | Complete | Complete |
| | after UV | | peeling | peeling | peeling | peeling | peeling |
| | exposure | | in 10 min | in 10 min | in 10 min | in 10 min | in 10 min |

*Dose on UV exposure after printing = 1830 mJ/cm²
*Time for immersion in pure water = 5 min

### [General Evaluation]

In the resist composition for pattern printing, which contains the binder, the filler, the thickener, the polyfunctional (meth)acrylate, and the titanium oxide having a photocatalytic activity and having an average particle diameter of 1 nm or more and 200 nm or less, and does not contain a photoinitiator, the printability, the etchant resistance, and the resist peelability, which are the basic performances of the resist, are favorably maintained, and the effect of suppressing the seepage is remarkably improved. In addition, Comparative Example 2 in which the photoinitiator is added, causes impairment of the peelability, which demonstrates an advantage of the Examples in which no photoinitiator is added purposefully.

1. A resist composition for pattern printing, comprising:
a binder component;
a filler component;
a thickener component; and
a polyfunctional (meth)acrylate, wherein the resist composition does not include a photoinitiator.

2. The resist composition for pattern printing according to claim 1, wherein a number of (meth)acrylate functional groups in the polyfunctional (meth)acrylate is 3 or more.

3. The resist composition for pattern printing according to claim 1, wherein a content of the polyfunctional (meth)acrylate is 0.1 parts by mass or more and 10 parts by mass or less with respect to 100 parts by mass of the resist composition for pattern printing.

4. The resist composition for pattern printing according to claim 1, wherein the resist composition for pattern printing further comprises a photocatalyst component.

5. The resist composition for pattern printing according to claim 4, wherein the photocatalytic component comprises titanium oxide having photocatalytic activity.

6. The resist composition for pattern printing according to claim 5, wherein an average particle diameter of the titanium oxide is 1 nm or more and 200 nm or less.

7. The resist composition for pattern printing according to claim 5, wherein a content of the titanium oxide is 0.01 parts by mass or more and 5 parts by mass or less with respect to total 100 parts by mass of the resist composition for pattern printing.

8. A method for producing a solar battery cell, comprising:
printing a resist pattern on a substrate having a semiconductor layer using a resist composition for pattern printing comprising a binder component, a filler component, a thickener component, and a polyfunctional (meth)acrylate, and not comprising a photoinitiator;
etching a part of the semiconductor layer and a part of the resist pattern; and
peeling off the residual resist pattern.

9. The method for producing a solar battery cell according to claim 8, wherein at least the resist pattern is irradiated with an actinic radiation after printing and prior to etching, or during etching.

10. The method for producing a solar battery cell according to claim 9, wherein the actinic radiation is an ultraviolet radiation or an electron beam.

11. The method for producing a solar battery cell according to claim 8, wherein the resist composition for pattern printing further comprises a photocatalyst component.

12. The method for producing a solar battery cell according to claim 9, wherein during etching, one or more selected from the group consisting of an aqueous acid solution, an aqueous alkaline solution, and water are brought into contact with the semiconductor layer and/or the resist pattern during or after the irradiation with the actinic radiation.

13. The method for producing a solar battery cell according to claim 8, wherein during peeling, an aqueous alkaline solution or an alcohol-containing aqueous solution is brought into contact with the semiconductor layer and/or the resist pattern.

14. The method for producing a solar battery cell according to claim 11, wherein the actinic radiation is applied even during or after peeling to remove a resist peeling residue.

15. A solar battery comprising a solar battery cell which is produced by the method for producing a solar battery cell according to claim 8.
